(19) United States
(12) Patent Application Publication
(10) Pub. No.: US 2013/0196390 A1
(43) Pub. Date: Aug. 1, 2013

(54) METHOD OF PRODUCING BIOFUEL, BIODIESEL, AND OTHER VALUABLE CHEMICALS

(71) Applicant: Eudes De Crecy, Gainesville, FL (US)

(72) Inventor: Eudes De Crecy, Gainesville, FL (US)

(21) Appl. No.: 13/580,537

(22) PCT Filed: Feb. 22, 2010
(86) PCT No.: PCT/US2010/024867

§ 371 Date: Feb. 22, 2013

(51) Int. Cl. C12P 7/64

(52) U.S. Cl. CPC C12P 7/649
USPC 435/134; 435/257.1; 435/289.1

(57) ABSTRACT

a method for producing a hydrocarbon based product (e.g., biofuel or biodiesel), a microorganism (e.g., algae) that can be cultivated and harvested for producing a hydrocarbon based product, a method for producing a hydrocarbon based product from a by-product of biodiesel production, and a microorganism that can be cultivated from a by-product of biodiesel production and harvested for producing hydrocarbon based product.

### BACKGROUND OF THE INVENTION

Petroleum is a term for “unprocessed” oil containing a mixture of hydrocarbons. Due to the different types of hydrocarbons that are present in petroleum, petroleum can be used as a starting material to obtain a variety of products.

The major classes of hydrocarbons in petroleum include paraffins (e.g., methane, ethane, propane, butane, isobutane, pentane, and hexane), aromatics (e.g., benzene and naphthalene), cycloalkanes (e.g., cyclohexane and methyl cyclopentane), alkenes (e.g., ethylene, butene, and isobutene), alkynes (e.g., acetylene, and butadienes).

Petroleum products fall into three major categories: 1) fuels such as motor gasoline and diesel fuel; 2) finished nonfuel products such as solvents and lubricating oils; and 3) feedstocks for the petrochemical industry such as naphtha and various refinery gases.

Petroleum is a non-renewable resource. As a result, many people are worried about the eventual depletion of petroleum reserves in the future. World petroleum resources have even been predicted by some to run out by the 21^{st} century (Kerr R A, Science 1998, 281, 1128).

This has fostered the expansion of alternative hydrocarbon products such as biofuel, also referred to as biodiesel, “bio”-hydrocarbon products, renewable hydrocarbon products, and fatty acid based products. Biofuel is a processed fuel derived from biological sources. For example, biologically produced lipids such as biomass oils derived from plants, algae, and animal fats have been used for biofuel (Johnson D, 1987, Overview of the DOE/SERI aquatic species program FY 1986 Solar Energy Institute, Colorado, incorporated herein by reference). Biomass oils can be used as fuels in a variety of ways: directly as boiler fuels, processed into biodiesel (methyl esters), or processed into “bio-distallates” via refinery technology (Tyson et al, Biomass Oil Analysis: Research Needs and Recommendations, National Renewable Energy Laboratory, June 2004, incorporated herein by reference). However, biodiesel is primarily used as an alternative diesel fuel.

Producing biofuel from microorganisms such as algae and bacteria has been touted as an efficient way to produce biodiesel, and other hydrocarbon based products. The advantage being that the land requirement for growing microorganisms is very small. Independent studies have demonstrated that microorganisms are capable of producing 30 times more oil per acre than the current crops now utilized for the production of biodiesel.

Biofuel obtained from microorganisms (e.g., algae and bacteria) is also non-toxic, biodegradable and free of sulfur. As most of the carbon dioxide released from burning biodiesel is recycled from what was absorbed during the growth of the microorganisms (e.g., algae and bacteria), it is believed that the burning of biofuel than from the burning of petroleum, which releases carbon dioxide from a source that has been previously stored within the earth for centuries. Thus, utilizing microorganisms for the production of biofuel may result in lower greenhouse gases such as carbon dioxide.

Some species of microorganisms are ideally suited for biofuel production due to their high oil content. Certain microorganisms contain lipids and/or other desirable hydrocarbon compounds as membrane components, storage products, metabolites and sources of energy. The percentages in which the lipids, hydrocarbon compounds and fatty acids are expressed in the microorganism will vary depending on the type of microorganism that is grown. However, some strains have been discovered wherein up to 90% of their overall mass contain lipids, fatty acids and other desirable hydrocarbon compounds (e.g., Botryococcus).

Lipid and other desirable hydrocarbon compound accumulation in microorganisms can occur during periods of environmental stress, including growth under nutrient-deficient conditions. Accordingly, the lipid and fatty acid contents of microorganisms may vary in accordance with culture conditions.

The naturally occurring lipids and other hydrocarbon compounds in these microorganisms can be transesterified to obtain a biodiesel. The transesterification of a lipid with a monohydric alcohol, in most cases methanol, yields alkyl esters, which are the primary component of biodiesel.

The transesterification reaction of a lipid leads to a biodiesel fuel having a similar fatty acid profile as that of the initial lipid that was used (e.g., the lipid may be obtained from animal or plant sources). As the fatty acid profile of the resulting biodiesel will vary depending on the source of the lipid, the type of alkyl esters that are produced from a transesterification reaction will also vary. As a result, the properties of the biodiesel may also vary depending on the source of the lipid. (e.g., see Schuchardt, et al, TRANSESTERIFICATION OF VEGETABLE OILS: A REVIEW, J. Braz. Chem. Soc., vol. 9, 1, 199-210, 1998 and G. Knothe, FUEL PROCESSING TECHNOLOGY, 86, 1059-1070 (2005), each incorporated herein by reference).

Glycerol (also named glycerin) is a by-product of biodiesel production. For every 1 ton of biodiesel that is manufactured, 100 kg of glycerol are produced. As the production of biodiesel increases, it follows that the production of glycerol increases. For example, in 1999 biodiesel glycerol accounted for just 7% of the glycerol market and in 2004 that figure had grown to 19%.

Originally, there was a valuable market for glycerol. However, with the increase in global biodiesel production, the market price for glycerol has crashed. Once considered a valuable by-product, glycerol is quickly becoming a waste product with an attached disposal cost.

Adding to the disposal cost of glycerol are the impurities that are also formed when the glycerol is created. Glycerol is usually purified by either concentrating the raw glycerol after subjecting the solution to filtration, distillation, an active charcoal treatment, ion-exchange treatment and/or other purification techniques. Alternatively, the raw glycerol may be concentrated and the concentrate may then be treated with an active charcoal treatment, ion-exchange treatment, or other treatment known to one skilled in the art.

However, these techniques are expensive and often unable to remove all of the impurities that may be present. This is because a number of impurities found in raw glycerol (e.g., 1,2-propanediol, 1,3-propanediol, 3-methoxy-1,2-propanediol, or 2-methoxy-1,3-propanediol) may have physical properties that are similar to glycerol (U.S. Pat. No. 6,288,287). Accordingly, additional purification steps are often required that are even more difficult and/or expensive to perform.

In view of the need for biofuel and predicted difficulties in handling the by-products of biofuel production, new ways to produce biofuel and to dispose of the by-products of biofuel are needed.

### SUMMARY OF THE INVENTION

This invention relates to a method for producing biofuel, a microorganism that can be cultivated and harvested for producing biofuel, a method for producing biofuel from a by-product of biofuel production, a microorganism that can be cultivated from a by-product of biofuel production and harvested for producing biofuel itself, a raw glycerol processing site/plant, and biodiesel manufacturing site/plant.

### BRIEF DESCRIPTION OF THE DRAWINGS

FIG. 1 is a flowchart illustrating a preferred arrangement of equipment for a biodiesel manufacturing site and process for producing biodiesel.

FIG. 2 is a flowchart illustrating a preferred arrangement of equipment for a biodiesel manufacturing site and process for producing biodiesel and other hydrocarbon products.

### DETAILED DESCRIPTION OF THE PREFERRED EMBODIMENTS

The present invention relates to a method for producing hydrocarbon based products such as biofuel, biodiesel, “bio”-hydrocarbon products, renewable hydrocarbon products, and fatty acid based products by cultivating and harvesting microorganisms (e.g., algae and bacteria) that express a compound (e.g., lipid) that can be used as or used to obtain hydrocarbon based products.

The microorganisms (e.g., algae and bacteria) utilized by the present invention may be evolutionarily modified to serve as an improved source of biofuel, biodiesel, and other hydrocarbon products An evolutionarily modified (EMO) microorganism is defined as a microorganism that has been modified by natural selection techniques.

The microorganism can be evolutionarily modified by a number of techniques (e.g., serial culture, continuous culture, or chemostat). However, the microorganisms are preferably produced as disclosed in PCT Application No. PCT/US05/05616, or U.S. patent application Ser. No. 11/508,286, each incorporated herein by reference. By cultivating a microorganism in this manner, beneficial mutations will occur to produce brand new alleles (i.e., variants of genes) that improve an organism's chances of survival and/or growth rate in that particular environment.

The microorganism (e.g., algae or bacteria) of the present invention can constitute a different strain, which can be identified by the mutations acquired during the course of culture, and these mutations, may allow the new cells to be distinguished from their ancestors' genotype characteristics. Thus, one can select new strains of microorganisms by segregating individuals with improved rates of reproduction through the process of natural selection.

Microorganisms can be evolutionarily modified in a number of ways so that their growth rate, viability, and utility as a biofuel biodiesel, or other hydrocarbon product can be improved. For example, microorganisms can be evolutionarily modified to enhance their ability to grow on a particular substrate.

The microorganisms (e.g., algae or bacteria) are preferably naturally occurring and have not been modified by recombinant DNA techniques. In other words, it is not necessary to genetically modify the microorganism to obtain a desired trait. Rather, the desired trait can be obtained by evolutionarily modifying the microorganism. However, even genetically modified microorganisms can be evolutionarily modified to increase their growth rate and/or viability of a modified by recombinant DNA techniques.

For example, algae can be cultivated for use as a biofuel, biodiesel, or hydrocarbon based product. Most algae need some amount of sunlight, carbon dioxide, and water. As a result, algae are often cultivated in open ponds and lakes. However, when algae are grown in such an “open” system, the systems are vulnerable to contamination by other algae and bacteria.

In this regard, the present invention preferably utilizes heterotrophic algae (Stanier et al, Microbial World, Fifth Edition, Prentice-Hall, Englewood Cliffs, N.J., 1986, incorporated by reference), which can be grown in a closed reactor.

While a variety of algal species can be used, algae that naturally contain a high amount of lipids, preferably, 15-90%, 30-80%, 40-60%, and 25-60% by dry weight of the algae is preferred. Prior to the work of the present inventor, algae that naturally contained a high amount of lipids and high amount of bio-hydrocarbon were associated as having a slow growth rate. Evolutionarily modified algae strains can be produced in accordance with the present invention that exhibit an improved growth rate.

The conditions for growing the algae can be used to modify the algae. For example, there is considerable evidence that lipid accumulation takes place in algae as a response to the exhaustion of the nitrogen supply in the medium. Studies have analyzed samples where nitrogen has been removed from the culture medium and observed that while protein contents decrease under such conditions, the carbohydrate content increases, which are then followed by an increase in the lipid content of the algae. (Richardson et al, EFFECTS OF NITROGEN LIMITATION ON THE GROWTH OF ALGAE ON THE GROWTH AND COMPOSITION OF A UNICELLULAR ALGAE IN CONTINUOUS CULTURE CONDITIONS, Applied Microbiology, 1969, volume 18 page 2245-2250, 1969, incorporated herein by reference)

Preferred types of algae that can be practiced with the present invention are Chlorophyta (Chlorella and Prototheca), Prasinophyta (Dunaliella), Bacillariophyta (Navicula and Nitzschia), Ochrophyta (Ochromonas), Dinophyta (Gyrodinium) and Euglenozoa (Euglena). These types of algae have already been shown to grow on refined glycerol and would satisfy the requirements of a Refined Glycerol Test.

Cyanobacteria may also be used with the present invention. Cyanobacteria are prokaryotes (single-celled organisms) often referred to as “blue-green algae.” While most algae is eukaryotic, cyanobacteria is the most common exception. Cyanobacteria are generally unicellular, but can be found in colonial and filamentous forms, some of which differentiate into varying roles. For purposes of the claimed invention, cyanobacteria are considered algae.

Chlorella protothecoides and Dunaliella Salina are species that have been evolutionarily modified, cultivated, and harvested for production of a biodiesel.

The following publications relate to growing different types of algae and then harvesting algae for the purpose of producing biodiesel are incorporated by reference herein:
Xu et al, HIGH QUALITY BIODESEL PRODUCTION FROM A MICROALGA CHLORELLA PROTHECOIDES BY HETEROTROPHIC GROWTH IN FERMENTERS, Journal of Biotechnology, vol. 126, 499-507, 2006,
Kessler, Erich, PHYSIOLOGICAL AND BIOCHEMICAL CONTRIBUTIONS TO THE TAXONOMY OF THE GENUS PROTOTHECA, III. UTILIZATION OF ORGANIC CARBON AND NITROGEN COMPOUNDS, Arch Microbiol, volume 132, 103-106, 1982,
Johnson D, 1987, OVERVIEW OF THE DOE/SERI AQUATIC SPECIES PROGRAM FY 1986 SOLAR ENERGY INSTITUTE,
Pratt et al, PRODUCTION OF PROTEIN AND LIPID BY CHLORELLA VULGARIS AND CHLORELLA PYRENOIDOSA, Journal of Pharmaceutical Sciences, volume 52, Issue 10, 979-984 2006, and
Sorokin, MAXIMUM GROWTH RATES OF CHLORELLA IN STEADY-STATE AND IN SYNCHRONIZED CULTURES, Proc. N.A.S, volume 45, 1740-1743, 1959.

A benefit in using algae is that a variety of components can be extracted from the algae during the process. For example, lipid that comes from the algae may comprise Omega-3 oil. This essential oil, “essential” means that the human body can not produce this oil, has tremendous health benefits for the heart, the brain, and the eyes and is required for proper fetal neural development. Omega-3 oil may also be used as a dietary supplement.

Selected algae can also be the sources of a wide range of chemical compounds such as phycocolloids used in industry, food technology and as pharmaceuticals. Other algae like Dunaliella accumulate high concentrations of carotenoids such as β-carotene, astaxanthin, and canthaxanthin. These carotenoids have wide application as natural colorants and antioxidants.

In a preferred embodiment, protein that naturally occurs in the algae may also be isolated and recovered during the process of cultivating and harvesting the algae for a biodiesel. The protein may be used as a food source for humans and other animals. Alternatively, the protein may be used as a nitrogen and/or carbon source for culturing algae or other microorganisms.

Aside from producing food additives or nutrients, algae, especially marine algae, are a rich source of bioactive compounds which may be utilized in medicinal and agricultural products. Detailed screening of micro-algae in the last 20 years has revealed a whole new range of molecules with antibiotic, antiviral and anticancer activities as well as anti-inflammatory, hypocholesterolaemic, enzyme inhibiting and many other pharmacological activities (Borowitzka, M. A., CHEMICALS FROM MICROALGAE, Ed. Z. Cohen, pp.313-352. Taylor & Francis: London (1999))

Bacteria can also be evolutionarily modified to produce a biofuel comprising lipids, hydrocarbons such as phytanyl and dibiphytanyl molecules and fatty acids that are naturally expressed and produced by the microorganisms.

A particularly desirable fatty acid that can be obtained is mycolic acid. Mycolic acids usable in the present invention include but are not limited to α-mycolic acids, methoxymycolic acids, ketomycolic acids, epoxymycolic acids, and mycolic acid wax esters containing a double bond or a cyclopropane ring with an internal ester group.

Mycolic acids are naturally produced by certain types of bacteria. As long as the bacteria are capable of expressing mycolic acid, the type of bacteria used to produce the mycolic acid is not essential. However, preferred bacteria for practicing the present invention include Corynobacterium, Nocardia, and Mycobacterium.

Corynobacterium produce mycolic acids referred to as Corynnomycolic acids, which typically contain 22-36 carbon atoms.

Nocardomycolic acids typically contain 44-60 carbon atoms and are produced by Nocardia bacterium.

Mycolic acids isolated from Mycobacterium are called mycolic acids or eumycolic acids. Mycolic acids produced by Mycobacterium generally have 60-90 carbon atoms. A description of the various forms of mycolic acids found in Mycobacterium may be found in a review by Minnikin D E et al. (Arch Microbiol 1984, 139, 225), incorporated herein by reference.

A preferred organism for producing mycolic acids is Mycobacterium smegmatis. Mycobacterium smegmatis, a Mycobacterium that is a non-pathogenic, is a fast-growing and non-fastiduous bacterium (L. G. Wayne, Kubica, and G. P.: The Mycobacteria. In: Holt, J. G., Sneath, P. H., Mair, N. S, Sharpe, M. E. (Eds.). Bergey's Manual of Systematic Bacteriology Vol. 2. The Williams Wilkins Co.; Baltimore, Md.: 1435-1457, 1986, incorporated herein by reference).

Thus, another preferred method for producing a biodiesel of the present invention comprises culturing and growing an evolutionarily modified bacteria for expressing a compound such as fatty acids, lipids, phytanyl, dibiphytanyl, and/or mycolic acid; optionally fractionating the bacteria in the culture to obtain a fraction containing the compound; optionally isolating and chemically treating (e.g., by transesterification) the compound from said fraction; and incorporating the biodiesel into an engine fuel.

A further advantage of the claimed invention is that the microorganisms can be adapted to produce a biofuel such as biodiesel, or other hydrocarbon based product from the by-products of biofuel production.

In a preferred embodiment, the method comprises culturing and growing a microorganism expressing a compound such as fatty acids, lipids, phytanyl, dibiphytanyl, mycolic acid, and/or other hydrocarbon compounds, with a culture medium comprising the by-products of biofuel production (e.g., raw glycerol); optionally fractionating the microorganism in the culture to obtain a fraction containing the compound; and optionally chemically treating (e.g., by transesterification) the compound from said fraction; and processing the resulting mixture into a biodiesel.

Raw glycerol is the by-product of a transesterification reaction comprising glycerol and impurities such as fatty acid components, oily components, acid components, alkali components, soap components, alcohol component (e.g., methanol or ethanol) solvent (N-hexane) salts and/or diols. Due to the number and type of impurities present in raw glycerol, microorganisms exhibit little to no growth on the raw glycerol itself. However, the microorganism (e.g., algae or bacteria) can be evolutionarily modified to utilize raw glycerol as a primary carbon source.

The initial test for determining whether a particular type of microorganism will be able to grow in the presence of raw glycerol is the Refined Glycerol Test. The Refined Glycerol Test comprises culturing the microorganism in a medium comprising refined glycerol. The medium utilized in the Refined Glycerol Test may or may not have another carbon source such as glucose. However, the medium in the Refined Glycerol Test must contain a sufficient amount of glycerol so that it can be determined that the microorganism exhibits a minimum metabolizing capacity of the microorganism. The medium preferably contains 10 ml-50 ml per liter of refined glycerol, 0.1 ml-100 ml per liter of refined glycerol, and 2 ml-15 ml per liter of refined glycerol.

If a positive result (i.e., the microorganism grows in the medium) is obtained with the Refined Glycerol Test, the microorganism can be evolutionarily modified to grow in a medium comprising raw glycerol. The culture medium preferably comprises 10-100% raw glycerol as a carbon source, 20-90% raw glycerol as a carbon source, 30-75% raw glycerol as a carbon source, 40-75% raw glycerol as a carbon source, or 50.01-55% raw glycerol as a carbon source. Indeed, some strains of microorganisms have been evolutionary modified to grow on a culture medium containing 100% raw glycerol.

Evolutionarily modified algae or bacteria are preferred microorganisms for producing biodiesel itself from the by-products of biodiesel production. An evolutionarily modified algae obtained in accordance with the claimed invention has a maximum growth rate at least 25%, preferably 50%, 75%, 100%, 200%, 25%-100%, 25%-100%, 50%-150%, 25-200%, more than 200%, more than 300%, or more than 400% greater than algae of the same species that has not been evolutionarily modified to utilize raw glycerol as a primary carbon source.

Accordingly, the invention is directed to a method for processing raw glycerol by obtaining a raw glycerol fraction; and processing the raw glycerol fraction by cultivating microorganisms comprising lipids, proteins, essential oils, bioactive compounds, and other hydrocarbons, wherein the microorganism is heterotrophic and evolutionarily modified to process raw glycerol as a primary carbon source; and wherein the microorganism has a maximum growth rate at least 25% greater than of the same species that has not been evolutionarily modified to utilize raw glycerol as a primary carbon source.

In a preferred embodiment, a method for processing raw glycerol includes the steps of cultivating and recovering a microorganism (e.g. algae) that has been evolutionary modified to grow on a culture medium comprising raw glycerol; fractionating the microorganism to obtain a first lipid fraction; and transesterifying the first lipid fraction with an alcohol to obtain an alkyl ester fraction and a second raw glycerol fraction, and recovering the alkyl esters for use as a biodiesel.

Additional process steps of washing and filtering the biodiesel may be used to recover the biodiesel.

In a preferred embodiment, the algae discussed above such as Chlorophyta (Chlorella and Prototheca), Prasinophyta (Dunaliella), Bacillariophyta (Navicula and Nitzschia), Ochrophyta (Ochromonas), Dinophyta (Gyrodinium) and Euglenozoa (Euglena) are selected as the microorganism to grow of raw glycerol.

Chlorella protothecoides and Dunaliella salina are some particular species that has been evolutionarily modified, cultivated, and harvested for production of a biodiesel from a by-product of biodiesel production.

Another preferred embodiment of the invention is a method for producing a biodiesel product from a by-product of biodiesel production by

obtaining a by-product of biodiesel production, the by-product of biodiesel production comprising raw glycerol;

growing a microorganism with the by-product of biodiesel production, wherein the by-product of biodiesel production comprises raw glycerol and raw glycerol is the primary carbon source;

isolating and recovering the microorganism from the growing step;

fractionating the microorganism from the growing step to obtain a first lipid fraction and optionally recovering additional components from the fraction such as protein, carbohydrates and other chemicals;

transesterifying the first lipid fraction with an alcohol to obtain alkyl esters as the biodiesel product and a second or further by-product of biodiesel production comprising raw glycerol; and

recovering the biodiesel product and optionally washing and/or filtering the biodiesel product.

Thus, a microorganism can be evolutionarily modified to grow on a culture medium comprising a by-product of transesterification. The microorganism itself can then be utilized to produce a biofuel.

While it is envisioned that the most important commercial use for microorganisms grown from the by-products of biodiesel production will be to use the microorganisms themselves for products such as biofuel, biodiesel, “bio”-hydrocarbon products, renewable hydrocarbon products, and fatty acid based products. The invention is not limited to this embodiment. For example, if the microorganism is an alga, the algae could be grown from the by-products of biofuel production and harvested for use as a food, medicine, and nutritional supplement.

The present invention also relates to a raw glycerol processing site and biodiesel manufacturing site. To process raw glycerol, a culture unit (e.g., a reactor or continuous culture machine) is required for growing an evolutionary modified microorganism (e.g., algae) that processes raw glycerol as a primary carbon source, wherein the culture unit is adapted for growing the microorganism under conditions to grow the microorganism under suitable conditions (e.g., heterotrophic conditions).

The biodiesel manufacturing site in accordance with the present invention preferably includes the following equipment:

a reactor unit for chemically treating or transesterifying a first lipid fraction with an alcohol (e.g., methanol) to obtain alkyl esters and a raw glycerol fraction;

a separator unit (e.g., filtration or centrifugation) to separate the alkyl esters from the raw glycerol fraction;

a culture unit (e.g., a reactor or continuous culture machine) for growing an evolutionary modified microorganism (e.g., algae) that processes raw glycerol as a primary carbon source, wherein the culture unit is adapted for growing the evolutionary modified microorganism;

a microorganism processing unit (e.g., a fractionating device for obtaining and isolating the naturally occurring lipids of the microorganism) for processing the microorganism grown in the culture unit to obtain further lipid fractions;

optionally a return unit (e.g., a pump) for sending the further lipid fraction to the reactor unit;

optionally a protein or other by-product from the recovery unit (e.g., a filtration or centrifugation device) for recovering protein or other specific compounds from the microorganism after the microorganism has been processed in the processing unit; and

optionally a biodiesel recovery unit (e.g., a washing device or filtration device) to clean or wash the biodiesel.

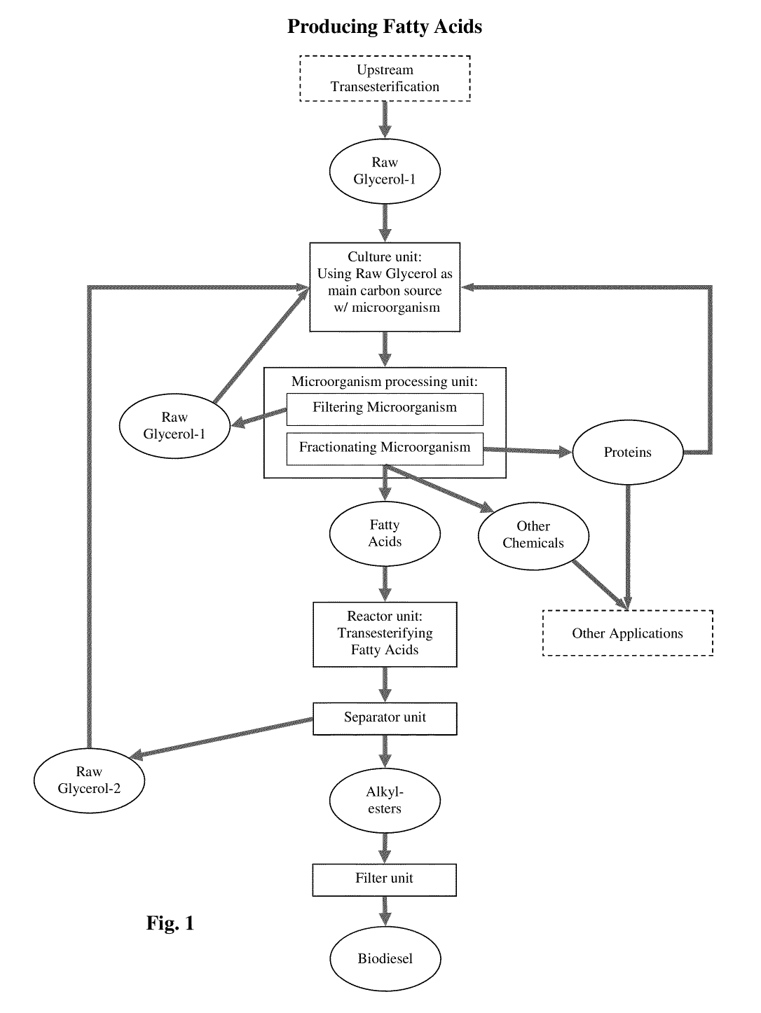

FIG. 1 exemplifies a preferred arrangement of equipment for a biodiesel manufacturing site and process for producing biodiesel. Raw Glycerol 1 is obtained from an upstream source.

The Raw Glycerol 1 is fed to a culture unit. The culture unit has an evolutionarily modified microorganism that utilizes Raw Glycerol 1 as a primary carbon source. The evolutionarily modified microorganism is grown and then filtered and/or fractionated to obtain fatty acids, proteins, and other chemicals.

The fatty acids are fed to a reactor unit to transesterify the fatty acids to produce alkyl esters. The alkyl esters may then be filtered and optionally processed to obtain a biodiesel product.

The proteins and other chemicals may be used in other applications. Alternatively, the proteins may be fed into the culture unit and used as supplemental source of nitrogen and/or carbon.

Unspent Raw Glycerol from growing the evolutionarily modified microorganism in the culture unit may be returned to culture unit for further processing.

In that the reactor unit carries out a transesterification reaction, a further source of raw glycerol is obtained, i.e., Raw Glycerol 2. The Raw Glycerol 2 may also be sent to the culture unit for processing.

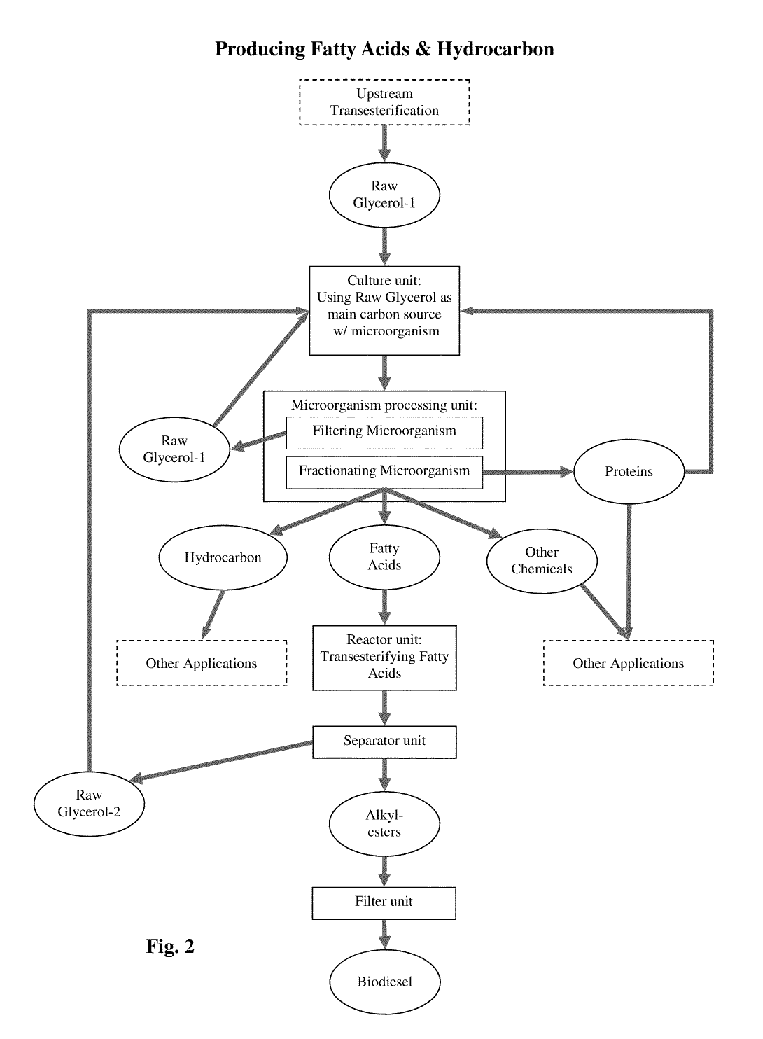

FIG. 2 shows a similar arrangement to FIG. 1 but indicates that hydrocarbons can be obtained from the process and utilized for other applications.

Once biofuel is obtained in accordance with the present invention, petroleum additives such as inorganic peroxides, organic peroxides, di-t-butylperoxide, alkyl nitrates, ethyl hexyl nitrate, amyl nitrate, and nitromethane may be added to the biodiesel. Petroleum alternatives such as ethanol, veggie oil, and other sources of biofuel may also be added.

The biofuel may be used directly or as an alternative to petroleum for certain products.

In another embodiment, the biofuel (e.g., biodiesel) of the present invention may be used in a blend with other petroleum products or petroleum alternatives to obtain fuels such as motor gasoline and distillate fuel oil composition; finished nonfuel products such as solvents and lubricating oils; and feedstock for the petrochemical industry such as naphtha and various refinery gases.

For example, the biofuel as described above may be used directly in, or blended with other petroleum based compounds to produce solvents; paints; lacquers; and printing inks; lubricating oils; grease for automobile engines and other machinery; wax used in candy making, packaging, candles, matches, and polishes; petroleum jelly; asphalt; petroleum coke; and petroleum feedstock used as chemical feedstock derived from petroleum principally for the manufacture of chemicals, synthetic rubber, and a variety of plastics.

In a preferred embodiment, biodiesel produced in accordance with the present invention may be used in a diesel engine, or may be blended with petroleum-based distillate fuel oil composition at a ratio such that the resulting petroleum substitute may be in an amount of about 5-95%, 15-85%, 20-80%, 25-75%, 35-50% 50-75%, and 75-95% by weight of the total composition. The components may be mixed in any suitable manner.

The process of fueling a compression ignition internal combustion engine, comprises drawing air into a cylinder of a compression ignition internal combustion engine; compressing the air by a compression stroke of a piston in the cylinder; injecting into the compressed air, toward the end of the compression stroke, a fuel comprising the biodiesel; and igniting the fuel by heat of compression in the cylinder during operation of the compression ignition internal combustion engine.

In another embodiment, the biodiesel is used as a lubricant or in a process of fueling a compression ignition internal combustion engine.

Alternatively, the biofuel may be further processed to obtain other hydrocarbons that are found in petroleum such as paraffins (e.g., methane, ethane, propane, butane, isobutane, pentane, and hexane), aromatics (e.g., benzene and naphthalene), cycloalkanes (e.g., cyclohexane and methyl cyclopentane), alkenes (e.g., ethylene, butene, and isobutene), alkynes (e.g., acetylene, and butadienes).

The resulting hydrocarbons can then in turn be used in petroleum based products such as solvents; paints; lacquers; and printing inks; lubricating oils; grease for automobile engines and other machinery; wax used in candy making, packaging, candles, matches, and polishes; petroleum jelly; asphalt; petroleum coke; and petroleum feedstock used as chemical feedstock derived from petroleum principally for the manufacture of chemicals, synthetic rubber, and a variety of plastics.

### EXAMPLE 1

Chlorella protothecoides was selected as the microorganism. Chlorella protothecoides has been shown to grow on refined glycerol and would satisfy the requirements of the Refined Glycerol Test. Chlorella protothecoides also naturally expresses a consistent amount of lipids.

The Chlorella protothecoides was then grown in a culture, wherein the carbon source comprises refined glycerol and raw glycerol. The Chlorella protothecoides is cultivated under conditions so that the amount of raw glycerol in the medium is slowly increased over a period of time so that the Chlorella protothecoides was evolutionarily modified to utilize raw glycerol as the primary carbon source.

The evolutionarily modified Chlorella protothecoides exhibited a higher maximum growth rate in a medium containing 100% raw glycerol than an unmodified strain of Chlorella protothecoides cultivated in a medium containing 100% refined glycerol.

A culture of evolutionarily modified Chlorella protothecoides was grown in a medium, wherein raw glycerol was the main carbon source. The Chlorella protothecoides was then isolated from the culture medium and optionally fractionated to obtain an extract comprising naturally expressed lipids in the Chlorella protothecoides.

### EXAMPLE 2

### Prophetic

The lipids obtained in Example 1 may then be transesterified to obtain alkyl esters. The alkyl esters may then be separated and purified by filtration techniques.

The alkyl esters may then be incorporated as a biodiesel in a distillate fuel oil composition comprising 50% fatty acids, and 50% diesel fuel by weight of the composition.

The distillate fuel oil composition may be used to fuel a compression ignition internal combustion engine.

### EXAMPLE 3

A culture of Dunaliella Salina algae is grown and evolutionary modified according to example 1.

### EXAMPLE 4

### Prophetic

The lipids obtained in Example 3 may then be transesterified to obtain alkyl esters. The alkyl esters may then be separated and purified by filtration techniques.

The alkyl esters may then be incorporated as a biodiesel in a distillate fuel oil composition comprising 50% fatty acids, and 50% diesel fuel by weight of the composition.

The distillate fuel oil composition may be used to fuel a compression ignition internal combustion engine.

### EXAMPLE 5

### Prophetic

An algal strain from Prototheca that naturally expresses a large amount of lipids is selected and found to grow on refined glycerol to satisfy the requirements of the Refined Glycerol Test.

Prototheca is then evolutionarily modified pursuant by continuous culture techniques. The Prototheca is initially grown in a culture, wherein the carbon source comprises refined glycerol and raw glycerol. The culture of Prototheca is then cultivated under conditions so that the amount of raw glycerol in the medium is slowly increased over time to evolutionarily modify the Prototheca so that the Prototheca is able to utilize raw glycerol as the primary carbon source.

The evolutionarily modified Prototheca has a higher maximum growth rate in a medium comprising 100% raw glycerol than an unmodified strain of Prototheca cultivated in a medium comprising 100% refined glycerol.

A culture of evolutionarily modified Prototheca is grown on raw glycerol as main carbon source until a sufficient amount of Prototheca is produced. The Prototheca may then be isolated from the culture medium and optionally fractionated to obtain an extract comprising lipids that are naturally expressed.

### EXAMPLE 6

### Prophetic

The lipids obtained in Example 5 may then be transesterified to obtain alkyl esters. The alkyl esters may then be separated and purified from the extract by filtration.

The alkyl esters may then be incorporated as a biodiesel in a distillate fuel oil composition comprising 50% fatty acids, and 50% diesel fuel by weight of the composition.

The distillate fuel oil composition may be used to fuel a compression ignition internal combustion engine.

What is claimed is:

1. An algae comprising:
lipids, and proteins, and
wherein said algae are heterotrophic and evolutionarily modified (EMO) to process raw glycerol as a primary carbon source.

2. The algae according to claim 1, wherein the algae has a maximum growth rate at least 25% greater than algae of the same species that has not been EMO to utilize raw glycerol as a primary carbon source, and wherein said algae was able to grow on glycerol in a refined glycerol test prior to being EMO.

3. The algae according to claim 1, wherein the algae is composed of 15-90% of lipids and/or hydrocarbon.

4. The algae according to claim 1, wherein the primary carbon source consists of raw glycerol.

5. The algae according to claim 1, wherein the algae has a maximum growth rate at least 50% greater than algae of the same species that has not been EMO to utilize raw glycerol as a primary carbon source.

6. The algae according to claim 1, wherein the algae are not modified by recombinant DNA techniques.

7. A method for processing raw glycerol, comprising
obtaining a first raw glycerol fraction; and
processing said first raw glycerol fraction by growing the algae according to claim 1 with said first raw glycerol fraction as a primary carbon source.

8. The method according to claim 7, further comprising
recovering said grown algae,
fractionating the grown algae to obtain a first lipid fraction; and
transesterifying the first lipid fraction with an alcohol to obtain an alkyl ester fraction and a second raw glycerol fraction, and
recovering the alkyl esters.

9. The method according to claim 8, further comprising recovering a protein fraction from the fractionating step.

10. The method according to claim 7, further comprising adding the second raw glycerol fraction to said first raw glycerol fraction so that the first and second raw glycerol fractions are processed in the processing step.

11. The method according to claim 7, further comprising filtering the alkyl esters to obtain a filtered biodiesel product.

12. The method according to claim 7, wherein said first raw glycerol fraction is obtained from a transesterification reaction.

13. The method according to claim 7, wherein the algae is composed of 15-90% of lipids and/or hydrocarbon.

14. The method according to claim 7, wherein the primary carbon source consists of raw glycerol.

15. The method to claim 7, wherein the algae have a maximum growth rate at least 25% greater than algae of the same species that has not been EMO to utilize raw glycerol as a primary carbon source.

16. The method according to claim 7, wherein the algae are not modified by recombinant DNA techniques.

17. A raw glycerol processing site, comprising a culture unit for growing evolutionary modified (EMO) algae that process raw glycerol as a primary carbon source, said culture unit comprising the algae according to claim 1 and raw glycerol.

18. A biodiesel manufacturing site, comprising:
i) a reactor unit for transesterifying a first lipid fraction with an alcohol to obtain alkyl esters and a raw glycerol fraction;
ii) a separator unit to separate the alkyl esters from the raw glycerol fraction;
iii) a culture unit for growing evolutionary modified (EMO) algae that utilizes the raw glycerol fraction as a primary carbon source, said culture unit comprising the algae according to claim 1 and raw glycerol fraction,
iv) an algae processing unit for processing the algae grown in said culture unit to obtain a second lipid fraction; and
v) a return unit for sending the second lipid fraction to the reactor unit.

19. The biodiesel manufacturing site according to claim 18, further comprising a protein recovery unit for recovering protein from said algae after said algae has been processed in the processing unit.

20. The biodiesel manufacturing site according to claim 18, wherein the culture unit has a growth chamber for growing the algae in a heterotrophic environment.

21. The biodiesel manufacturing site according to claim 18, further comprising a filter unit for recovering and filtering the alkyl esters to obtain a biodiesel product.

22. A method for producing a biodiesel product from a by-product of biodiesel production, comprising the steps of:
obtaining a by-product of biodiesel production, said by-product of biodiesel production comprising raw glycerol;
growing the algae according to claim 1 with said by-product of biodiesel production, and wherein the raw glycerol is the primary carbon source;
isolating and recovering the algae from said growing step;
fractionating the algae from said growing step to obtain a first lipid fraction;
transesterifying the first lipid fraction with an alcohol to obtain alkyl esters as the biodiesel product and a second by-product of biodiesel production comprising raw glycerol; and
recovering the biodiesel product, and optionally other hydrocarbon products, fatty acids products, and, and proteins.

23. The method according to claim 22, wherein said first by-product of biodiesel production is obtained from a transesterification reaction.

24. The method according to claim 22 , further adding the second by-product of biodiesel production to said first by-product of biodiesel production in said growing step.

25. The method according to claim 22, further comprising filtering the alkyl esters to obtain a biodiesel product.

26. The method according to claim 22, further comprising recovering a protein fraction or other chemical compound from the fractionating step.
